(19) United States
(12) Patent Application Publication
(10) Pub. No.: US 2009/0287166 A1
(43) Pub. Date: Nov. 19, 2009

(54) INTEGRATED DEVICE FOR ISCHEMIC TREATMENT

(75) Inventor: Diane Mai Huong Dang, Palo Alto, CA (US)

Correspondence Address: SAWYER LAW GROUP PC, 2465 E. Bayshore Road, Suite No. 406, PALO ALTO, CA, 94303, US

(73) Assignee: CNA Tek Inc., Palo Alto, CA (US)

(21) Appl. No.: 12/123,425

(22) Filed: May 19, 2008

(51) Int. Cl. A61M 5/00; A61M 25/00

(52) U.S. Cl. 604/265; 604/528

(57) ABSTRACT

A system and method in accordance with the present invention provides a infusion catheter that is flexible, has such a smooth traction and a low profile to minimize break up of the obstruction when crossing it, can access distal vasculature quickly, is easy to use and readily to be implemented in the conventional PCI. Another object is to provide a method that can be performed within a short period of time and employs the catheter described herein to infuse a therapeutic agent distally to the obstruction before it is removed as a means of reducing reperfusion injury, protecting distal vasculature and microcirculation, preserving myocytes, reducing infarct size and ischemic damages in the heart, brain, lung, liver, kidney and limb. Another object is to provide complementary feature options to the infusion catheter and the aspiration catheter to improve the speed and quality of the vessel clearance.

### FIELD OF THE INVENTION

This present invention generally relates to catheters and more particularly to intravascular catheters used to protect the distal vasculature, to improve tissue survival, and to accelerate clearance of acute blocked vessels.

### BACKGROUND OF THE INVENTION

Clinical evidence has shown that Percutaneous Coronary Intervention (PCI) or interventional/endovascular procedure in general is the superior treatment method for acute ischemic disease in the heart and in the brain. Within the first three (3) hours after onset of pain or other symptoms of acute myocardial infarct, PCI and intravenous thrombolysis appear to be equally effective in reducing infarct size and mortality. For the majority of patients, who arrive in the hospital after the three hour window, suffer a more serious attack such as ST elevation MI (STEMI), or are contra-indicated or non-responsive to thrombolysis, PCI is superior in reducing short term mortality, nonfatal infarction, and stroke. PCI enables a rapid clearance of the occluded vessel by local thrombolysis, mechanical fragmentation or rheolytic thrombectomy. Aspiration of thrombus using a guiding catheter or an aspiration catheter is a popular method not only for coronary vessels, but also for neurovascular and peripheral vessels.

Despite an achievement of a high percentage of complete recanalization or normal Thrombolysis in Myocardial Infarction flow grade 3 (TIMI3) flow, myocardium salvage and infarct size reduction are less than expected. Potential causes for the poor outcome using these techniques are distal embolization, blockage of microcirculation and capillaries, microvascular constriction and spasm, less than optimal myocardial blush grade, reperfusion injury, endothelial cell injury and dysfunction.

As thrombectomy and distal protection can remove thrombus and debris in more than 75% of patients, these treatments are expected to reduce distal embolization, therefore reducing infarct size and enhancing survival. However, there was no improvement in the main clinical end points such as infarct size or ST segment resolution in most randomized trials. Manual thrombectomy/thrombus aspiration using an aspiration catheter is effective in reducing thrombus load and debris, but at the same time, suction appears to induce powerful vasoconstriction and trigger the release of vasoactive, inflammatory molecules. Obviously, prevention of distal embolization is not enough or the conventional technique may trigger side effects that counteract the benefits of aspiration in the ischemic tissues and distal vasculature.

Targeted delivery of pharmaceutical agents such as adenosine infusion in AMISTAD II, intracoronary infusion of hyperbaric oxygen solution in AMIHOT II, or local intravascular rapid cooling in COOL MI and ICE-IT suggested that modifications of PCI procedures can limit reperfusion injury and reduce infarct size, particularly in the more severe anterior infarction. However, improvements remain in most cases statistically non-significant. Under this circumstance, an extension in treatment time, a need for additional equipment, and an increase in the risk and cost associated with these treatments strongly inhibit their implementation in clinical practice.

Intracoronary infusion is often performed by using i) a guiding catheter, ii) an over-the-wire (OTW) infusion catheter or microcatheter, or iii) through the guidewire lumen of an over-the-wire balloon catheter. The last two types of catheters can move along the guidewire to access individual coronary vessels, such as left main LAD, LCX, and RCX. To allow infusion to occur through the lumen, the guidewire must first be removed from the OTW lumen that extends all the way from the proximal hub to the distal tip. Handling of OTW balloon catheters is more cumbersome, often requires two operators and the use of extra long guidewire. Any change of the infusion target afterward requires time-consuming re-insertion and advance of the guidewire. In order to keep the guidewire in place, some OTW catheters have a much larger lumen that allows fluid infusion in the presence of the guidewire but they are quite bulky and used mainly for diagnostic purposes.

In contrast to OTW design, the rapid exchange (RX) or monorail design allows keeping the guidewire in place and maintaining the advantage of reaching any target quickly is. One operator can handle both RX catheter and the guidewire. As illustrated in the example of catheters supporting dual guidewires, the first guidewire can be easily exchanged and provides enough tracking support for the whole catheter when it is positioned in the short monorail. A separate OTW lumen allows the second guidewire to access a branch vessel or to support parallel guidewire or guidewire exchange for tackling chronic total occlusion or bifurcation. As these multi-functional probing catheters combine the tracking advantage of a rapid exchange catheter with an OTW lumen, for some chronic total occlusion treatment cases, physicians use them to visualize distal vessels or to inject vasodilator distally in a rescue effort to fight against severe spasm or life-threatening situations like no-reflow. Because these catheters are designed to support handling of two guidewires or probes in their dual-lumen construction at the distal part, their distal part is bulky. Another type of monorail catheter having dual lumen at the distal part is the aspiration catheters. The OTW aspiration lumen is even larger to create efficient suction, increasing the total profile further.

Another type of rapid exchange catheter having infusion capability is based on a balloon catheter. Holes or slits on the balloon surface allow drug to leave the fully inflated balloon and to diffuse into the surrounding for restenosis and local thrombolysis treatment. As the inflated balloon contacts firmly with the vessel wall or surrounding thrombus, the contact between fully inflated balloon surface and the surrounding vessel wall or thrombus controls the amount of drug release.

Current catheters and treatment methods that aim at protecting the distal vasculature and reducing ischemic tissues are performed by injecting therapeutic agents only after 1) the balloon of the catheter that delivers drugs is inflated or 2) after the blood flow is re-established. As such, they are associated with several inherent drawbacks. In the first scenario, the large profile and pressure generated by inflated balloons break up or push thrombus towards the vessel wall, generating a shower of emboli in the distal vasculature and blocking microcirculation system. These emboli practically stop blood flow in the microcirculation system and therefore stop the therapeutic agent from reaching its target. In the second scenario, reperfusion injury occurs as soon as blood flow is re-established, making it much more difficult or even impossible to reverse reperfusion injury with subsequent infusion of therapeutic agents

Accordingly, what is needed is a system and method that addresses the above-identified issues. The present invention addresses such a need.

### SUMMARY OF THE INVENTION

A system and method in accordance with the present invention provides a infusion catheter that is flexible, has such a smooth traction and a low profile to minimize break up of the obstruction when crossing it, can access distal vasculature quickly, is easy to use and readily to be implemented in the conventional PCI. Another object is to provide a method that can be performed within a short period of time and employs the catheter described herein to infuse a therapeutic agent distally to the obstruction before it is removed as a means of reducing reperfusion injury, protecting distal vasculature and microcirculation, preserving myocytes, reducing infarct size and ischemic damages in the heart, brain, lung, liver, kidney and limb. Another object is to provide complementary feature options to the infusion catheter and the aspiration catheter to improve the speed and quality of the vessel clearance.

### BRIEF DESCRIPTION OF THE DRAWINGS

FIG. 1 is a drawing of the heart in which the vessels are enlarged to illustrate the treatment.

FIGS. 2A-2C shows the perpendicular and longitudinal cross sections (or cross section view and side elevation view) of the infusion catheter.

FIGS. 3A-3D illustrates shapes of the infusion lumen and the guidewire lumen.

FIGS. 4A-4E are diagrams of the treatment using the infusion catheter in combination with an aspiration catheter as an example to clear the vessel.

FIG. 5 illustrates the main steps as illustrated in FIGS. 4A-4E.

FIGS. 6A-6E are diagrams of the treatment using the infusion catheter which has an emboli pulling wire frame and polymer basket attached near the tip.

FIG. 7 illustrates the main steps as illustrated in FIGS. 6A-6E.

FIGS. 8A-8E illustrate an aspiration catheter with a flexible polymer sleeve to improve the efficiency of aspiration.

### DETAILED DESCRIPTION OF THE INVENTION

This present invention generally relates to catheters and more particularly to intravascular catheters used to protect the distal vasculature, to improve tissue survival, and to accelerate clearance of acute blocked vessels. The following description is presented to enable one of ordinary skill in the art to make and use the invention and is provided in the context of a patent application and its requirements. Various modifications to the preferred embodiment and the generic principles and features described herein will be readily apparent to those skilled in the art. Thus, the present invention is not intended to be limited to the embodiment shown but is to be accorded the widest scope consistent with the principles and features described herein.

### Definitions

Percutaneous Coronary Intervention (PCI), interventional or endovascular procedure has the same meaning for the treatment of the heart.

Other words for PCI or similar approach: interventional procedures, interventional cardiology, interventional neurology, endovascular procedure.

Revascularzation and recanalization: re-establish blood flow in the block vessel.

Reperfusion often refers to interventional approach.

Thrombus, clot, emboli can have a similar meaning. Emboli can be blood clot or plaque fragments.

Occlusion, obstruction or blockage is caused by thrombus, or thrombus built at the plaque rupture site, thrombus or emboli migrating from another site.

Distal refers to the part of the device that goes farthest into the body or to the direction toward smaller vessel lumen. Proximal refers to the part of the device closer to the end which remains outside of the body during the procedure. Proximal also refers to the direction toward larger vessel lumen.

Rapid exchange and monorail are used interchangeable. Injection is usually a short infusion and may be used interchangeable.

Agents serve diagnostic purpose (contrast agent for X-ray or ultrasound imaging), and therapeutic purpose (cardioprotective agent, neuroprotective agent, thrombolytic agent, anti-platelet, vasodilator, ion channel and mitochondrial permeability transition pore (mPTP) active agents, anti-inflammatory agent, anti-oxidants, radical scavenger, anesthetic agent, enzymes; gene silencing agents siRNA and anti-sense mRNA, gene delivery vehicles; stem cells; growth factors, or conditioned media enriched with growth factors).

Physical removal of an obstruction in the blood vessel includes ablating by laser or ultrasound, mechanical cutting, shaving, scraping, pressurizing, pushing to the vessel wall, pulling, and aspirating or suction. Thrombolysis is the process in which thrombus or blood clot is dissolved by biological (enzymes) or chemical means.

### Description

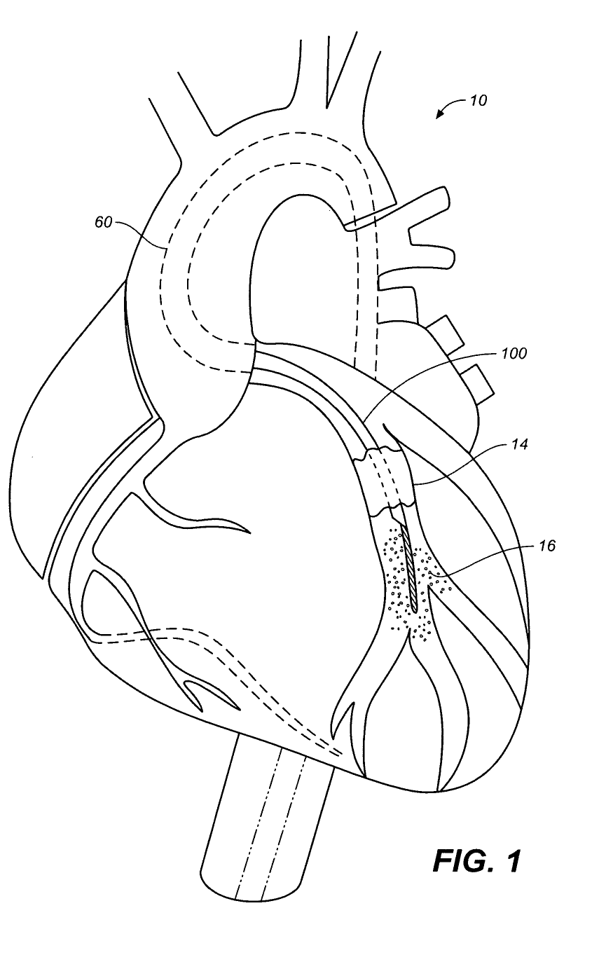

FIG. 1 is a drawing of the heart 10 in which the vessels are enlarged to illustrate the treatment. A large obstruction 14 is seen in the left anterior descending artery (LAD). A guiding catheter 60 with its tip is seating at the ostium. The monorail infusion catheter 100 is advanced along the guidewire crossing the obstruction 14. The obstruction 14 remains mainly intact. Distal protective treatment is performed by injecting the agent 16 distally to the obstruction 14, supplying agent specifically to the distal vasculature and ischemic tissues beyond the obstruction, in the very short time before the obstruction is dissolved by local thrombolytic agent or removed by another means.

### Rapid Exchange RX Infusion Catheter

The main advantage of endovascular procedures over the medical thrombolysis therapy in the treatment of acute ischemic obstruction is the almost instantaneous clearance of the occluded vessel or recanalization via thrombus removal. While the traditional interventional devices and procedures address recanalization, the device and method in accordance with the present invention enable a unique combination of interventional and medical therapy that provides clinical improvement beyond a simple recanalization.

It is well known that both destructive and protective changes naturally occur in the tissues, cell gaps, cell membrane, and within cells under extended ischemic conditions. Rapid reperfusion stops major deteriorations and keep ischemic areas at bay but it also interferes with body defensive mechanisms. As an example, ischemia induces blood-brain barrier alteration, disruption, up to breakdown. Reperfusion occurs when the blood-brain barrier is not fully recovered is believed to be a precursor to the more lethal hemorrhagic event following an ischemic stroke.

As a natural mechanism to reduce stroke-induced injury, adenosine levels increase in the brain up to 100-fold following a stroke,. Adenosine, when bound to A1 receptor, inhibits undesired ion flows through the membrane of cells exposed to ischemia. Adenosine, when bound to A2 receptors, produces positive effects such as vasodilation and inflammation inhibition. Instantaneous reperfusion brought by PCI limits ischemic regions from growing, but also washes out and eliminates the protective effect of adenosine more quickly.

Oxygen depletion forces tissues to obtain energy through anaerobic glycolysis, a process which results in the accumulation of lactic acid. Such acidic conditions can lead to apoptosis and aggravate ischemic injury. On the other hand, mitochondrial permeability transition pores (mPTP) that do not open in acidic milieu during ischemia quickly open as acidosis is relieved and pH increases after reperfusion. Opening of mPTP leads to collapse of the mitochondrial transmembrane potential, cessation of ATP production, mitochondrial content depletion and subsequently cell death. For cells and tissues suffering reversible injury, a rapid change the microenvironment associated with instantaneous reperfusion may lead to irreversible injury and permanent loss.

While impairment of membrane permeability and function as well as restricted blood flow in the ischemic areas are destructive, they offer a very short timely window in a strictly limited space to maximize the efficacy of a therapeutic treatment. Limited flow, in combination with a reduction in barrier, gap, and membrane integrity in the ischemic areas enhance the retention, and uptake of therapeutic agents, gene and cell therapy vehicles to an unsurpassed level. However, any treatment via intracoronary drug delivery only can take advantage of this unique opportunity if the distal vasculature and the microcirculation system are not blocked by emboli.

A catheter in accordance with the present invention addresses and solves exactly these challenges.

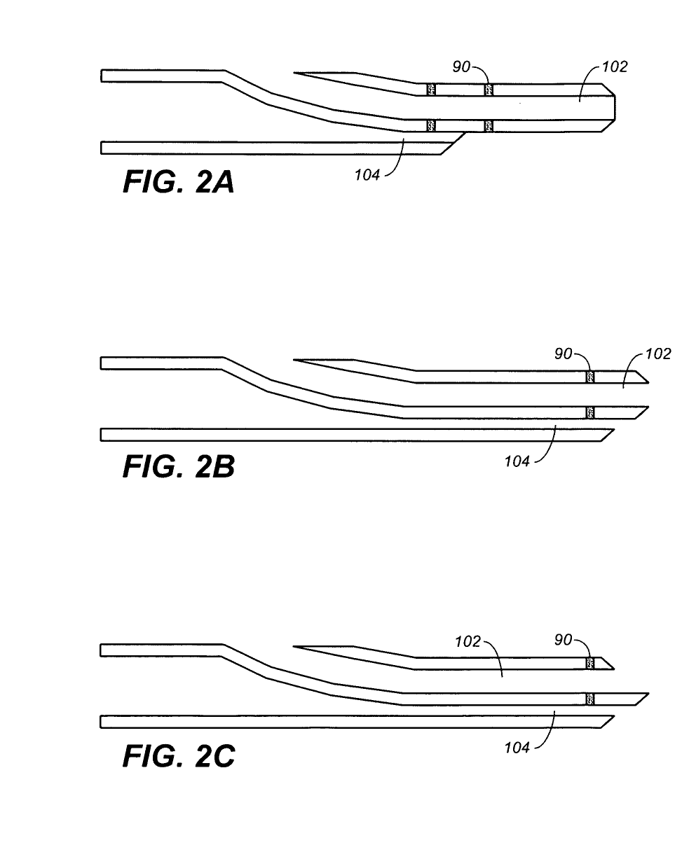

FIGS. 2A-2C show the perpendicular and longitudinal cross sections (or cross section view and side elevation view) of the distal part of infusion catheter 100 in accordance with an embodiment. The larger but shorter lumen 102 is for the guidewire. The narrower lumen 104 can serve as infusion lumen

The catheter 100 can cross the obstruction at the minimal risk of obstruction break up and inject solutions of therapeutic agents distally to it quickly, and precisely due to i) support of the guidewire at all time, ii) very low crossing profile, iii) tapered tip design, and iv) hydrophilic coating on the outside surface of the distal tip of the catheter 100 to reduce friction. The monorail design allows the catheter 100 to firmly advance or re-tract along the guidewire placed in the lumen 102. The inner diameter ID of the monorail lumen 102 can be, for example, 0.0005″-0.004″, preferably 0.0005-0.0020″ larger than the OD the guidewire, commonly at 0.014″ for coronary guidewire, or 0.010″ for neurovascular guidewire. The monorail lumen 102 is just big enough for the guidewire 80 to slide straight inside

A lubricious, inner lining of the monorail lumen 102 is made of fluorinated polymers such as polytetraethylen (PTFE), fluorinated ethylene-propylen copolymer (FEP), fluorinated polyether (FP), PTFE dispersion in another polymer such as polyimide or nylon. High density polyethylene (HDPE) or graphite powder in a polymer matrix are another options for the lubricious lining. Although they are not as lubricious as fluorinated polymers, their surface is not as smooth. Therefore the point-type contact on these surfaces creates no more resistant than surface-type contact on fluorinated polymer surface. The lubricious lining facilitates a smooth guidewire movement inside the guidewire lumen 102, but still thin enough not to affect the flexibility of the distal part of the catheter 100. The strength of the monorail lumen 102 wall is contributed by a thicker wall made of a variety of materials such as nylon, Pebax, polyurethane, polyethylene, and polyimide. The wall can be extruded or solvent-cast directly over the inner lining or over a tie layer to increase the attachment between the wall and inner lining.

The monorail lumen 102 from the most distal tip to a guidewire exit notch should be long enough to support the advancing of the catheter 100, but short enough to facilitate guidewire, catheter handling, and infusion. This length usually varies from 5-30 cm, preferably 15-25 cm. Within this length, the guidewire lumen 102 and infusion lumen 104 are essentially parallel to each other, and the diameter of the infusion lumen 104 is also narrower to minimize the distal crossing profile, and consequently, to minimize any break up of the obstruction when the catheter 100 crosses the obstruction. Proximally to the guidewire exit notch, the diameter of the infusion lumen 104 is enlarged. For a common catheter length of 130-150 cm, about 20 cm of the infusion lumen 104 is narrow while the majority 100-130 cm is large to keep the infusion pressure low. Low infusion pressure makes it easier to inject agents. Low infusion pressure also puts less stress/strain damages on biological active agents such as cells and enzymes, so they are able to retain more biological activities when they reach the target. Moreover, the wall thickness can be significant less for low pressure infusion than for high pressure infusion and contributes positively for a flexible distal shaft and a low crossing profile.

A radiopaque marker 90 made of a heavy, noble metal such as gold, platinum, palladium or platinum/iridium alloy can be attached very near the tip of the infusion lumen 104. The marker may be around the monorail lumen 102 distally or proximally to the tip of the infusion lumen 104. Since the marker is placed outside of the monorail lumen 102, it does not disturb the guidewire movement. Physicians can use the radiopaque marker 90 at the tip of the infusion lumen 104 to determine the exact location where a therapeutic or diagnostic agent is delivered.

The proximal shaft of the catheter 100 is the single lumen, enlarged part of the infusion lumen 104. The outer diameter of the proximal infusion lumen 104 can be larger, equal or smaller than the distal crossing profile of the infusion catheter 100. This single lumen part often contains a stainless steel braiding embedded in a single polymer matrix or in a multilayer matrix, such as Pebax, Nylon, polyurethane, polyethylene or polyimide over stainless steel. Stainless steel tubing or, for low pressure infusion, a polymer tubing such as polyimide tubing is another alternative. A braided tubing often experiences good push and torque ability, good kink resistance, and can be built with a gradual transition in stiffness. A tapered stiffening wire or supporting mandrel can be added between the single lumen shaft and the distal, narrower infusion lumen 104 to improve the push and torque transfer as well as a smooth transition in stiffness of catheter 100.

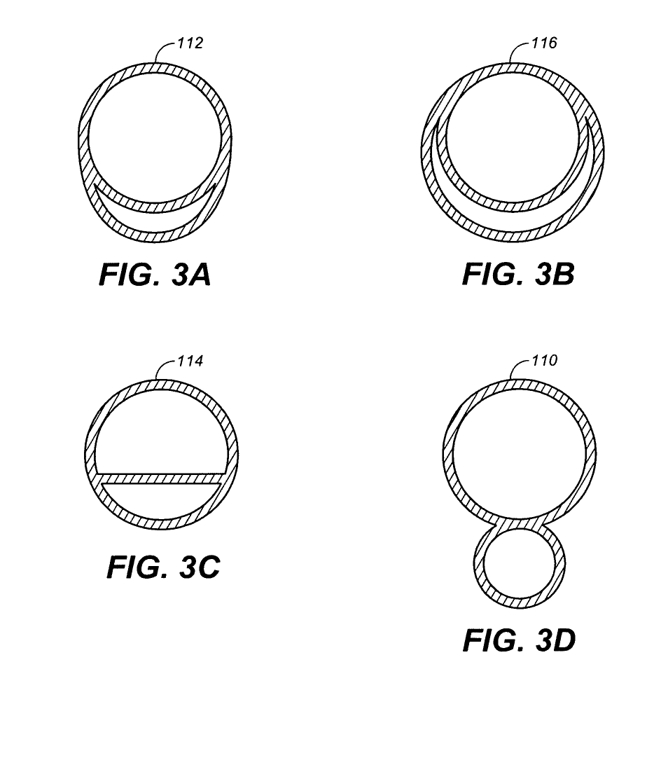

The guidewire lumen 102 and the smaller, infusion lumen 104 can form an “8” shape 110 (FIG. 3D) or a “smiling-face” shape 112 or 116 at (FIG. 3A and FIG. 3B) or a “split circle” shape (FIG. 3C) 114 in the distal part of the catheter 100 as shown in FIG. 2A. These configurations can be formed by multi-lumen extrusion, solvent-casting, heat-shrink or lamination around a mandrel and the inner tubing, or bonding of inner and outer tubing. The bond can be created by heat, infrared radiation, ultrasound welding, laser welding, or adhesives. A pre-treatment of the bonding surface by vacuum or atmospheric plasma, corona discharge, or chemical activation can improve bonding strength between inner and outer tubing. Similar surface activation methods can be used to prepare the surface for a hydrophilic coating if needed.

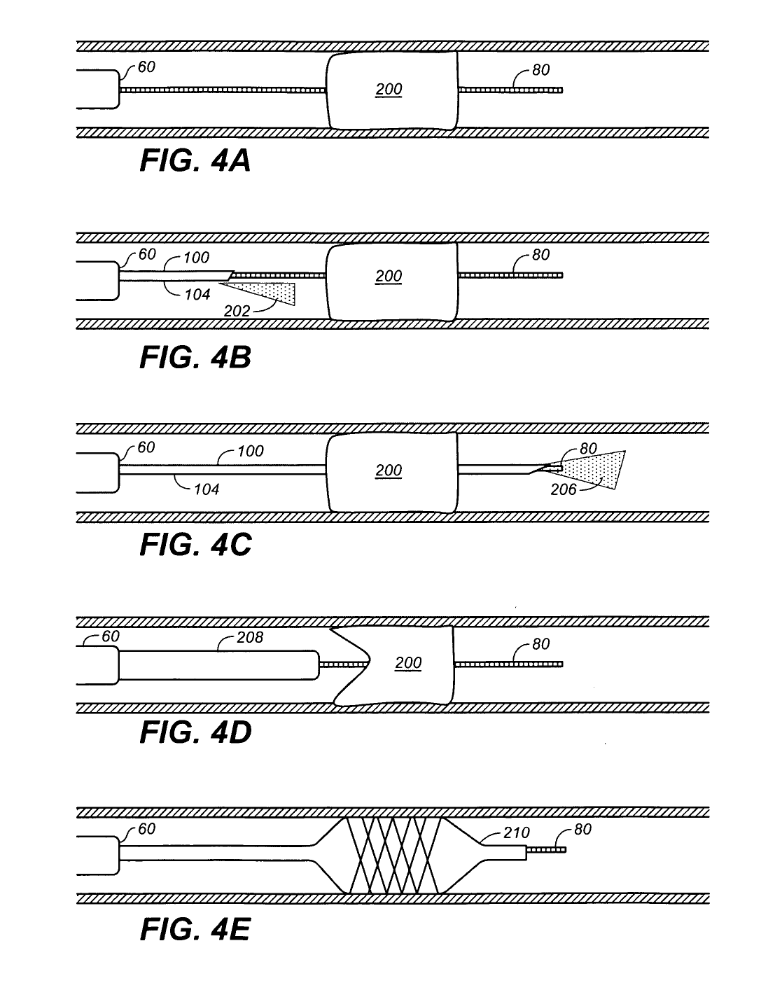

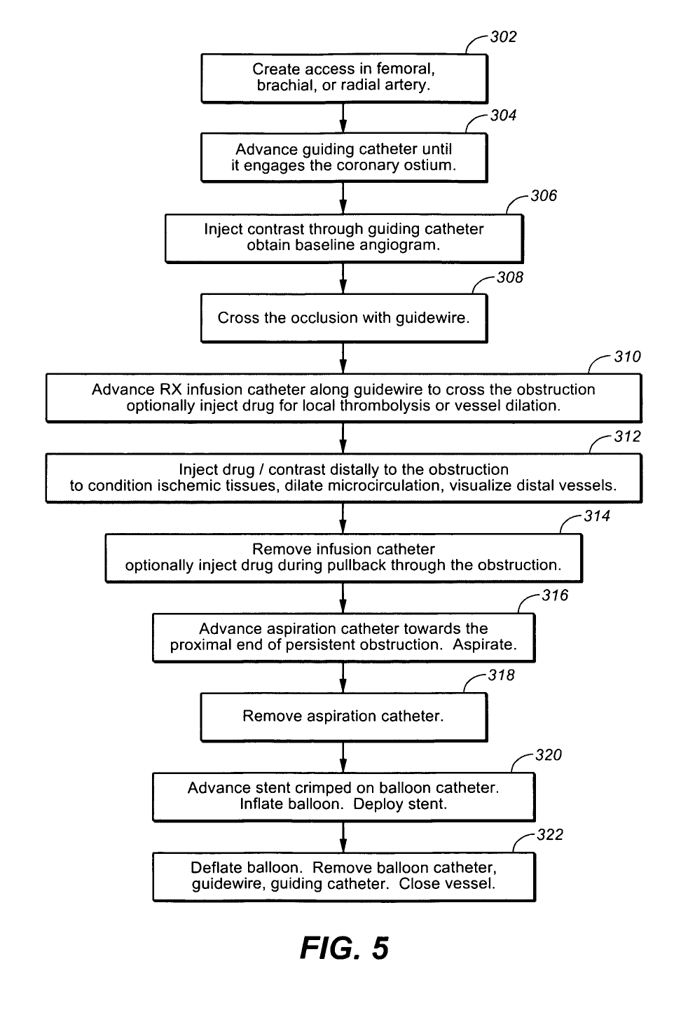

FIGS. 4A-4E is a sequential diagram illustrating the treatment using the infusion catheter in combination with an aspiration catheter as an example to clear the coronary vessel. FIG. 5 is a flow chart illustrating the treatment. Referring to FIGS. 4A-4E together, first access in femoral, brachial, or radial therapy is created, via step 302. Then, guiding catheter 60 is advanced until it engages the coronary ostium, via step 304. Next, contrast is injected through guiding catheter 60; and baseline angiogram is obtained, via step 306. Thereafter the obstruction 200 is crossed with guidewire 80, via step 308. The RX infusion catheter 100 is then advanced along guidewire to cross the obstruction, with thrombolytic drug 202 optionally injected near or at the obstruction 200 to initiate or enhance local thrombolysis, via step 310. A protective drug or contrast 206 is injected distally to the obstruction to condition ischemic tissues, dilate microcirculation and visualize distal vessels, via step 312. The infusion catheter 100 is then removed, more drug is injected for local thrombolysis and vessel dilation during pullback through the obstruction if necessary, via step 314. If the obstruction 200 persists, the aspiration catheter 208 is advanced along the guidewire 80 towards the proximal end of obstruction and the thrombus is aspirated, via step 316. A small and flexible guiding catheter can be used sometimes as an aspiration catheter to remove an obstruction in a large vessel. The aspiration catheter 208 is removed, via step 318. The stent crimped on the balloon catheter 210 is advanced to the obstructed site, the balloon is inflated and the stent is deployed, via step 320. Finally the balloon is deflated, balloon catheter 210 and guidewire 80, and the guiding catheter 60 are removed; and the vessel is closed, via step 322.

Referring back to FIGS. 2A-2C, The very distal tip of the catheter 100 is designed to cross obstruction 200 with the least resistance and volume placement in order to minimize any break up of the obstruction. First of all, the distal tip is built as small as possible, but still enable infusion of therapeutic agents, partly due to the enlarged proximal infusion lumen. For an 0.014″ guidewire, the guidewire lumen 102 is 0.015″-0.018″. The distal diameter of infusion lumen 104 is at about 0.005″-0.012″. For a wall thickness between 0.001 and 0.005″, preferably between 0.002-0.003″, the total crossing profile of a “smiling-face” monorail infusion catheter for a 0.014″ guidewire is between 0.028-0.036″, preferably between 0.028″-0.033″. Using thinner wall tubing can reduce the crossing profile further. In neurovascular applications, where blood vessels are much smaller, the guidewire lumen 102 is reduced to accommodate a smaller, 0.010″ guidewire, resulting in a significant reduction of the crossing profile. Vice versa, the guidewire lumen 102 will be enlarged to accommodate a 0.018″ or 0.035″ guidewire in peripheral applications.

When the distal diameter of the infusion lumen 104 gets smaller, injection pressure increases. Connecting the proximal end of the infusion lumen 104 with a pump can provide better dosing and can keep the lumen 104 under slow, continuous drip of heparin/saline to prevent thrombus formation and clogging at the tip. Using a one-way valve is another way to keep the full length of the infusion lumen 104 filled with heparin/saline and free of thrombus after the initial flushing. A manifold attachment to the proximal end of the infusion lumen 104 enables an easy switch between different infusion solutions: heparin/saline for flushing, radiopaque solution for visualization, vasodilator to relieve spasm, or protective agents can be utilized to reduce reperfusion injury.

A hydrophilic or very smooth infusion lumen 104 can greatly reduce the pressure to inject aqueous solutions of agents, and potentially can decrease the distal diameter of the infusion lumen to less than 0.005″. Hydrophilic coating or using a layer of a biocompatible polymer with a hydrophilic surface active group such as OH, COOH, SO3H (sulfonated), copolymer, blend, surface grafting, coating with polyvinylpyrrolidone, polyvinylalcohol, polyethylene glycol, polymerized polyethylene glycol acrylate, sulfonated polyethylene glycol, sodium polyvinyl sulfonate, heparin, etc. create a hydrophilic, non-thrombogenic surface with low resistance for fluid flow/infusion and catheter movement. Surface active polymers containing hydrophilic groups, such as block, graft and end-group modified polymers also result in hydrophilic surface upon wetting. These hydrophilic modifiers can be applied, for example by a solvent-casting or a co-extrusion process, as a thin inside and outside liners on surface of the main polymer in the wall of the infusion lumen 104.

In addition to the small total crossing profile and the hydrophilic coating on the outside, the thermally shaped or laser grinded tapered tip facilitates forward movements of the infusion catheter 100 and its ability to cross obstruction by gently pressing soft thrombus to the wall instead of breaking it, and so minimizing the risk of distal embolization. Furthermore, as protective agents can be delivered directly and selectively to the vessels and tissues affected by the obstruction, it is possible to achieve therapeutic efficacy at a lower total dose and at the same time, to minimize unwanted side effects of systemic delivery. In addition, this infusion catheter is especially suitable to deliver agents that may not be effective by other delivery methods because they are not stable in blood or are easily absorbed by other blood components. By limiting both the delivery time and the contact with blood before reaching the target, a sensitive agent has the best chance to retain its full potency.

The optional injection of thrombolytic agent near or at the obstruction via step 310 gives the more gentle, local thrombolysis another chance before the initiation of an active removal process of persistent obstruction via step 316. The protective/revival treatment by distal injection of drug via step 312 takes places concurrently with the local thrombolysis. During this short period, existing obstruction initially acts as a natural barrier to reduce blood flow, leading to an increase in the retention, uptake and efficacy of protective therapeutic agents in the distal vasculature and tissues, before it is dissolved. A small, single lumen, OTW infusion catheter or an OTW balloon catheter can also be used to cross the obstruction for distal drug delivery after the guidewire is removed. In this case, the whole treatment takes a longer time because at every infusion target, the guidewire must be removed to clear the infusion lumen and re-inserted before the catheter is moved to the next target or is withdrawn.

In summary, by reducing the crossing profile of the monorail infusion catheter to the minimum and by reducing the friction through hydrophilic coating and tapered tip design, the infusion catheter can reach the distal vasculature at the minimal risk of thrombus fragmentation. This catheter can move easily with the guidewire remains in place, therefore it can deliver different drugs to different targets in a short time. This short pre-reperfusion treatment can prepare the distal vasculature and areas at risk better for reperfusion. The treatment can positively tip the balance in the area at risk and turn reversible injury back to normal. The catheter and the method described herein can be used to treat ischemic events in all organs that have been shown to suffer from reperfusion injury, such as heart, brain, lung, liver, kidney and limb.

In addition to the therapeutic applications, the capability of this infusion catheter for easy delivery of agent to distal target site, its small profile and flexibility that do not stretch or irritate vessel makes it a good device for diagnostics and vessel visualization, especially distal vessels, with less contrast agent, and consequently, less toxicity for the kidney. This dual lumen, small profile catheter can also be used as support catheter for steerable or small guidewires. Additional mechanical support provided by the guidewire lumen wall and improved distal visibility thanks to contrast injected through the infusion lumen facilitate the forward movement and lesion crossing of the guidewire itself. Other potential uses of this small profile, monorail infusion catheter includes the local delivery of embolization or chemotherapy in cancer patients where increasing precision and decreasing contact surface are likely to improve efficacy and to reduce side effects of the treatments.

### Distal Wire-Frame Embolic Pulling Basket and Filter (Embolic Protection and Removal Device)

A modification of the monorail infusion catheter contains a shape-memory wire-frame basket attached on the outside surface of the guidewire lumen and near the distal tip in order to improve the removal of thrombus or other solid fragments. A “shape-releasing” wire for the wire frame basket runs through the infusion lumen. During delivery, the wire frame basket is stretched along the catheter. At the target site, the wire frame is released to resume the disk shape. A porous or non-porous baggy polymer membrane completely covers the distal part of the wires for more than 50% of the total length, and shields vessel wall from irritation or injury caused by thin wires. The proximal ends of the wires are exposed, allowing the wires to cut through soft thrombus when being pulled back towards an aspiration or a guiding catheter. For delivery, the membrane is folded or wrapped around the wires to reduce friction caused by the wire frame,

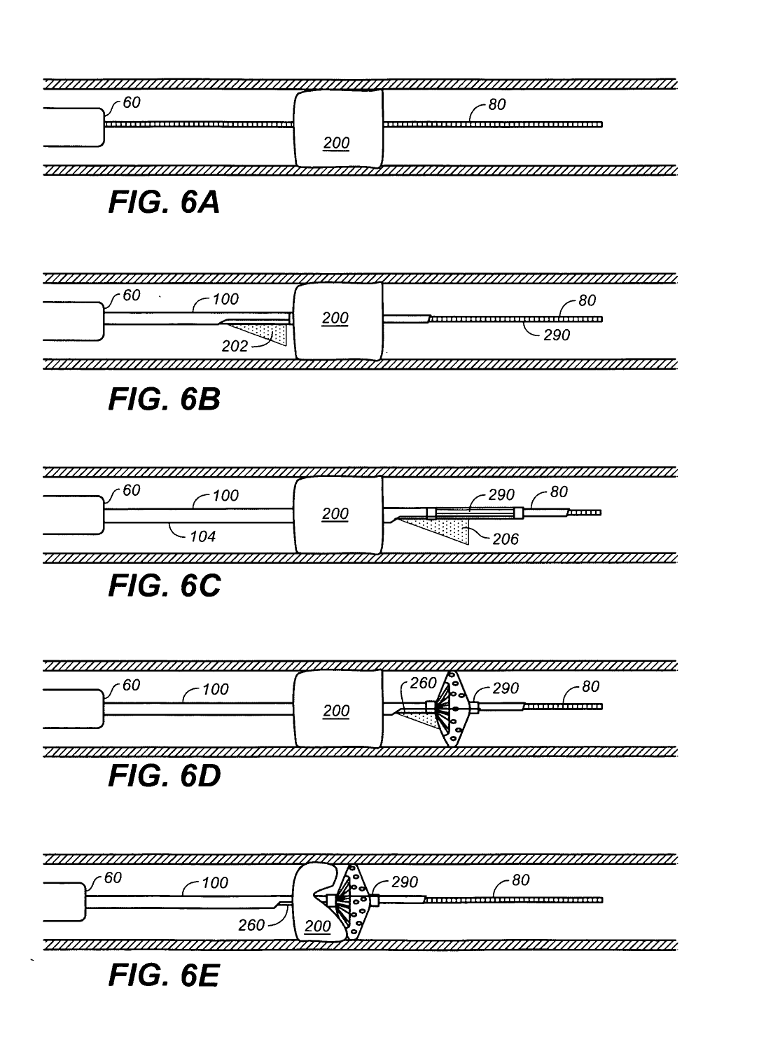

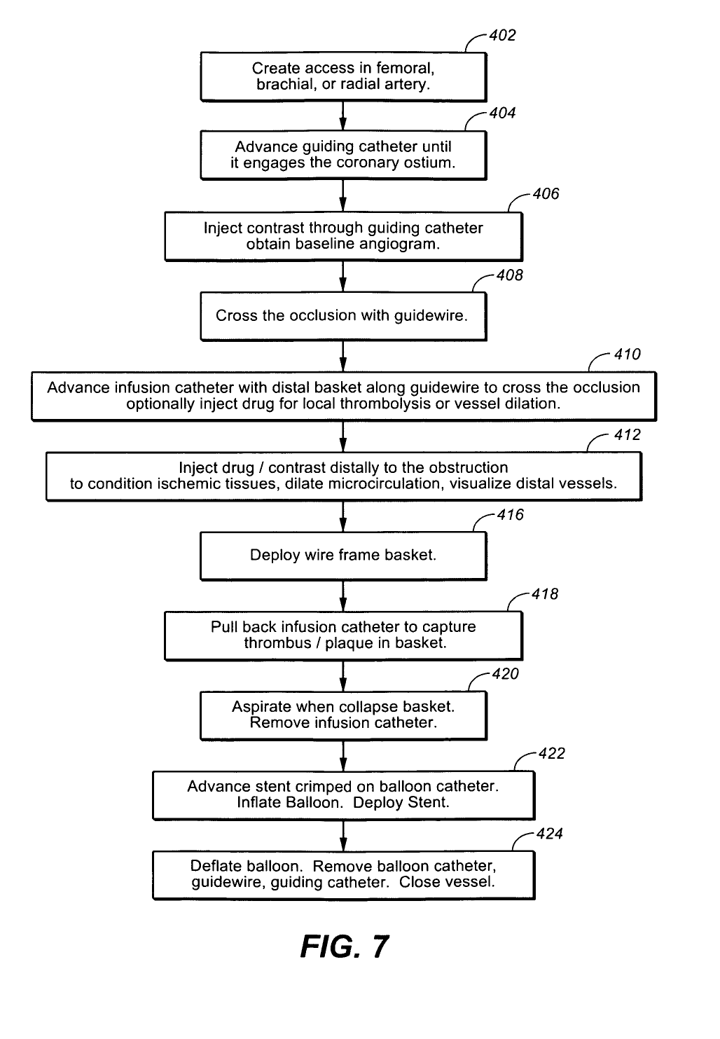

FIGS. 6A-6E are sequential diagrams of the treatment using the infusion catheter which has an emboli pulling wire frame and polymer basket attached near the tip. FIG. 5 is a flow chart that illustrates the treatment. Referring to FIGS. 6A-6E and 7 together, access in femoral, brachial, or radial artery is created via step 402. The guiding catheter 60 is advanced until it engages the coronary ostium, via step 404. Thereafter, contrast is injected through guiding catheter 60; and baseline angiogram is obtained, via step 406. The obstruction 200 is crossed with guidewire 80, via step 408. Then the infusion catheter 100 with distal basket 290 is advanced along guidewire to cross the obstruction 200, with optional infusion of thrombolytic agent or vessel dilating agent 202 via step 410. The drug and contrast 206 is injected distally to the obstruction 200 to condition ischemic tissues, dilate microcirculation, and visualize distal vessels, via step 412. The wire frame basket 290 is deployed, via step 416. The infusion catheter 100 is pulled back to capture thrombus/plaque in the basket 290; and optionally a drug is injected for local thrombolysis, or the vessel, via step 418. The vessel is aspirated when the basket is collapsed, and infusion catheter 100 is removed, via step 420. Much like FIG. 3A, the stent crimped on balloon catheter is advanced; the balloon is inflated; and stent is deployed, via step 422. Finally the balloon is deflated, the balloon catheter, guidewire and guiding catheter are removed; and the vessel is closed, via step 424

### This Wire Frame Design Can Have the Following Features:

Individual wires of the frame are laser cut from tubing or are available as pre-finished wires. The orientation of the wires can be strait or preferably in spiral pattern to increase the contacting surface and plaque removal effect. The wire is preferably round, small and soft wires in the distal part, including the largest diameter of the disk, for easy adjustment of the disk diameter and of the disk shape in non-circular vessels.

Rectangular wires with the proximal part become twisted when resuming the disk shape can better remove plaque, calcified lesion, old thrombus. A combination of the two types of wires is preferred: smaller, softer rounded segments in the distal half to reduce irritation for vessel; larger, stronger rectangular twisted segments in the proximal end to improve plaque removal at the center. By attaching the wire frame and basket 290 on the extended monorail lumen 102 of the infusion catheter 100, as shown in FIGS. 6A-6E, the infusion catheter 100 now has the option to actively remove thrombus and plaque that can not be removed easily by local thrombolysis.

The wire frame can undergo laser cutting, electropolishing, and shape setting. For the rectangular wires in the proximal end of the frame, chemical etch and passivation techniques are preferred to retain their cutting edge. Optionally, a thin and ductile gold layer can be electroplated on nitinol to increase the radiopacity, visibility of the wire frame basket in PCI.

The intact or porous polymer basket or membrane around the wire frame is formed from a balloon, a polymer thin film or a polymer filter membrane, preferably with 100 um pores. The basket is fixed to the distal end of the wire frame and wraps around the distal part of stretched wires as means to reduce friction and to limit particle generation during delivery and crossing through the obstruction. In one embodiment the polymer basket has a hydrophilic coating in the outside to reduce friction when it crosses the obstruction. At the target, the release of the shape-releasing wire 260 let the wire frame resume its pre-shaped disk and open the basket to retain obstruction fragments when the infusion catheter 100 is pulled back.

Alternatively, the wire frame and basket 290 can be attached directly on a guidewire to reduce the profile if needed, as alternative to current distal embolic protection device.

### Aspiration Catheter:

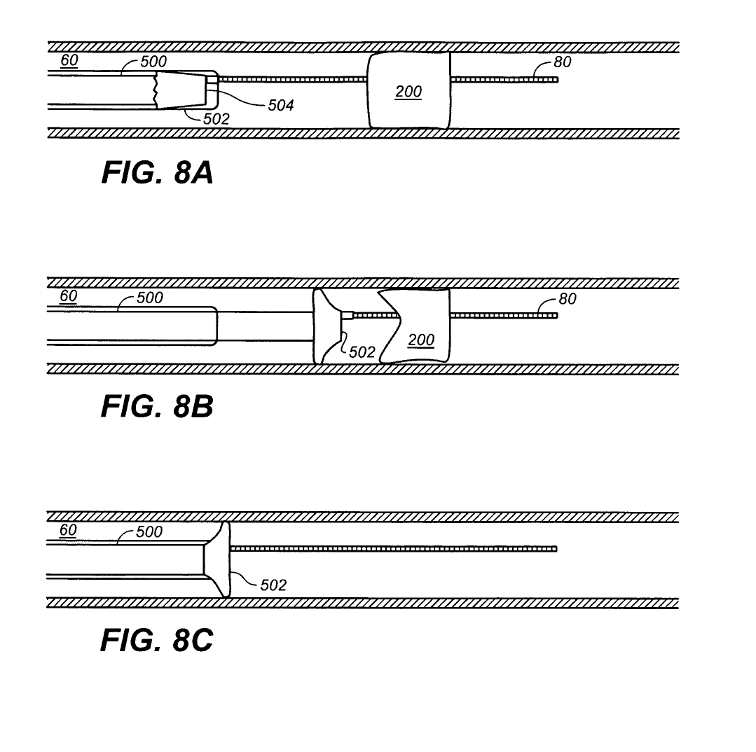

FIGS. 8A-8E illustrates an aspiration catheter 500 with a soft, flexible polymer sleeve 502 to improve the efficiency of aspiration. The sleeve 502 is released when the aspiration catheter tip 504 exits the guiding catheter 60 or when protective sheath is removed. The soft sleeve 502 is larger than the diameter of vessel lumen, allowing the sleeve to adapt its opening to the actual circular or non-circular vessel lumen. By reducing the blood flow from the proximal towards the distal direction, an opened sleeve 502 can improve aspiration. When the aspiration catheter 500 is pulled back into the guiding catheter, the sleeve 502 reverses its shape and squeezes to the center allowing an easy removal.

The polymer sleeve may contain a radiopaque agent such as tungsten, bismuth powder or iodine compound mixed in the polymer matrix. Alternatively, the polymer can be attached to or embed a soft platinum or platinum alloy frame or ring to allow visualization of the sleeve position relative to the aspiration tip and the obstruction. The shape of the sleeve depends on the tip of the aspiration catheter. A disc-shaped sleeve works well with a straight catheter tip. Preferably, the aspiration catheter has a slanted tip to enlarge the suction cross section. In this case, the sleeve has an elliptical shape. The soft polymer sleeve 502 has a hydrophilic coating on the vessel side to minimize trauma to the endothelial cell lining inside the vessel lumen. Optionally, a reversible, higher swelling coating on one surface of the polymer sleeve 502 facing the catheter side may be used to support the umbrella shape of the opened sleeve. A shape-memory platinum, platinum alloy, or nitinol ring at the catheter tip can be used to facilitate the opening of the sleeve when the aspiration catheter tip exits the guiding catheter.

Although the present invention has been described in accordance with the embodiments shown, one of ordinary skill in the art will readily recognize that there could be variations to the embodiments and those variations would be within the spirit and scope of the present invention. Accordingly, many modifications may be made by one of ordinary skill in the art without departing from the spirit and scope of the appended claims.

What is claimed is:

1. An infusion catheter comprising:
a guidewire lumen; and
an infusion lumen coupled to the guidewire lumen for providing a drug infusion to a vessel containing obstruction, wherein the distal profile of the infusion catheter is such that a breakup of the obstruction in the vessel is minimized when the infusion catheter is extended therethrough.

2. The infusion catheter of claim 1 wherein the distal diameter of the infusion lumen is less than the diameter of the guidewire lumen.

3. The infusion catheter of claim 1 wherein the distal crossing profile is 0.036″ or less.

4. The infusion catheter of claim 1 wherein the infusion lumen diameter is nonuniform.

5. The infusion catheter of claim 1 wherein the infusion lumen serves as a channel for a wire controlling the opening of embolic pulling bracket.

6. The infusion catheter of claim 1 wherein the guidewire lumen includes a lubricious inner lining.

7. The infusion catheter of claim 1 wherein part of the surface of the infusion lumen is hydrophilic

8. The infusion catheter of claim 1 wherein the guidewire lumen is long enough to support the advancing of the catheter but short enough to facilitate infusion, guidewire and catheter handling.

9. The infusion catheter of claim 1 wherein the profile of the guidewire lumen and the infusion lumen form an “8” shape.

10. The infusion catheter of claim 1 wherein the profile of the guidewire lumen and the infusion lumen form a “smiling face” shape.

11. The infusion catheter of claim 1 wherein the profile of the guidewire lumen and the infusion lumen form a “split circle” shape.

12. The infusion catheter of claim 1 wherein a radiopaque marker is located at a distal end of the infusion lumen.

13. The infusion catheter of claim 1 wherein the catheter serves as support for the guidewire placed inside the guidewire lumen.

14. A method for acute ischemic treatment comprising:
crossing an obstruction within a vessel with a catheter; and
infusing drug distally to the obstruction by the catheter.

15. The method of claim 14 wherein the catheter has an overall profile in which when the catheter is extended through the obstruction the break up of the obstruction is minimized.

16. The method of claim 14 wherein infusing drug distally to the obstruction within a vessel benefits cells and tissues connected to that vessel.

17. The method of claim 14 wherein the infusing step is preferably less than 10 minutes.

18. The method of claim 14 wherein a thrombolytic agent is injected when the catheter passes through the obstruction.

19. The method of claim 14 wherein the obstruction is physically removed after the distal infusion.

20. The infusion catheter of claim 14 wherein a radiopaque marker is located at a distal end of the infusion lumen.

21. The method of claim 14 to treat ischemic disease in heart, brain, lung, kidney, limb.

22. The method of claim 14 wherein a guidewire is removed from the catheter tip before the infusion.

23. The method of claim 14 which includes injecting contrast to ensure an infusion tip passes the obstruction.

24. An infusion catheter comprising:
a guidewire lumen; and
an infusion lumen coupled to guidewire lumen for providing a drug infusion, wherein the distal diameter of the infusion lumen is narrower than the diameter of the guidewire lumen.

25. The infusion catheter of claim 24 wherein the distal diameter of the infusion lumen is equal to or less than the diameter of a guidewire entering the guidewire lumen.

26. The infusion catheter of claim 24 wherein the infusion lumen diameter is nonuniform.

27. The infusion catheter of claim 24 wherein the infusion lumen serves as a channel for a wire controlling the opening of embolic pulling bracket.

28. The infusion catheter of claim 24 wherein the guidewire lumen includes a lubricious inner lining.

29. The infusion catheter of claim 24 wherein part of the surface of the infusion lumen is hydrophilic.

30. The infusion catheter of claim 24 wherein the guidewire lumen is long enough to support the advancing of the catheter but short enough to facilitate infusion, guidewire and catheter handling.

31. The infusion catheter of claim 24 wherein the profile of the guidewire lumen and the infusion lumen form an “8” shape.

32. The infusion catheter of claim 24 wherein the profile of the guidewire lumen and the infusion lumen form a “smiling face” shape.

33. The infusion catheter of claim 24 wherein the profile of the guidewire lumen and the infusion lumen form a “split circle” shape.

34. The infusion catheter of claim 24 wherein a radiopaque marker is located at a distal end of the infusion lumen.

35. The infusion catheter of claim 24 wherein the catheter serves as support for the guidewire placed inside the guidewire lumen

36. An aspiration catheter comprising a flexible sleeve at the tip of the aspiration catheter.
